(19) United States
(12) Patent Application Publication
(10) Pub. No.: US 2023/0084724 A1
(43) Pub. Date: Mar. 16, 2023

(54) METHOD FOR PRODUCING HYDROXYAPATITE-BIOGLASS MATERIALS, SAID MATERIALS AND PRODUCTS THEREOF

(71) Applicants: UNIVERSIDADE DO PORTO, Porto (PT); BIOSCKIN, MOLECULAR & CELL THERAPIES S.A., Maria (PT)

(72) Inventors: Ana MAURÍCIO, Porto (PT); Ana BRANDÃO, Maria (PT); Maria SIMÕES, Maria (PT); Carla MEIRELES, Maria (PT); Ana BARBOSA, Maria (PT); Luís ATAíDE, Porto (PT); Carla MENDONÇA, Porto (PT)

(21) Appl. No.: 17/911,435

(22) PCT Filed: Mar. 6, 2021
(86) PCT No.: PCT/IB2021/051889

§ 371 Date: Sep. 14, 2022

(30) Foreign Application Priority Data
Mar. 20, 2020 (PT) 116179

(51) Int. Cl. A61L 27/42; C03C 4/00; C03C 3/247; A61L 27/56

(52) U.S. Cl. CPC A61L 27/425; C03C 4/0007; C03C 3/247; A61L 27/56; C03C 2204/00; A61L 2430/02

(57) ABSTRACT

The present invention relates to a method for producing hydroxyapatite-bioglass macroporous material, to said materials, and to medical devices thereof.

The method comprises a step of preparation of an aqueous suspension of hydroxyapatite and bioglass with a porogenic agent, and subsequent sintering to achieve a macroporous biomaterial.

The macroporous structure of these materials enhances blood vessels and bone cells migration, allowing bone growth through the interior of the bone substitute, thereby increasing the rate of formation of new bone at the site of implantation. Therefore, these materials are advantageously used to produce medical devices, such as bone grafts that resemble the mineral phase of natural bone showing improved mechanical strength and osteoconductivity.

The biomaterials of the present invention are applicable in the medical area, in particular in bone regeneration and reparation techniques as bone grafts.

### TECHNICAL DOMAIN OF THE INVENTION

The present invention relates to a method for producing hydroxyapatite-bioglass materials, said materials and products thereof, such as medical devices.

The method comprises a step of preparation of an aqueous suspension of hydroxyapatite and bioglass with a porogenic agent, and subsequent sintering to achieve a macroporous biomaterial.

The macroporosity structure of these materials enhances blood vessels and bone cells migration, allowing bone growth through the interior of the bone substitute, thereby increasing the rate of formation of new bone at the site of implantation.

Therefore, these biomaterials are advantageously used to produce medical devices, such as bone grafts that resemble the mineral phase of natural bone showing improved mechanical strength and osteoconductivity.

The biomaterials of the present invention are applicable in the medical area, in particular in bone regeneration and reparation techniques as bone grafts.

### BACKGROUND OF THE INVENTION

The bone is a complex mineralized tissue that exhibits rigidity and strength, while maintaining a certain degree of elasticity, existing in two forms, the primitive bone and lamellar bone. The first class is an immature bone that is formed during embryonic development, cicatrisation and fracture healing processes, tumours and metabolic diseases. Its structural organization is random. The lamellar bone is a more mature bone that gradually replaces the primitive bone, representing the major class of bone in the adult skeleton and possessing a well-organized structure. It is constituted by cortical bone (external bone region) and trabecular bone (internal bone region).

The cortical bone is characterized by cylindrical canals (osteons), united by a rigid tissue matrix which is essentially composed by hydroxyapatite. Collagen cylindrical fibres (the main organic component of bone) fill the pores (190-230 μm) of this kind of bone. The inorganic matrix of the cortical bone consists of a structure with approximately 65% interconnective porosity. On the other hand, the trabecular bone differs from the cortical bone by showing further empty spaces and non-cylindrical pores filled with collagen. Trabecular bone pores, in the range of 500-600 μm are larger than cortical bone pores. Therefore, it becomes apparent that due to its intrinsic complex structure, the bone is one of the most difficult tissues to mimic.

Currently, average life expectancy is twice as high as in the beginning of the 20^{th} century, resulting in a progressive tissue functionality loss. Of note, the incapacity associated to orthopaedic degeneration clinical challenges, which is considered a major social problem in modern society's aged populations.

Currently, bone is the second most transplanted material to the human body, only preceded by blood. Bone defects resulting from trauma, tumour resection, fracture non-union and congenital malformations are common clinical problems.

The consensual gold standard graft remains the autologous graft, consisting of bone collection in one site and transplantation to another site of the same individual. These grafts possess limitations concerning amount availability, as well as, the invasive nature of the harvest procedure. Due to their autologous origin, these grafts eliminate the risk of infection transmission (Human Immunodeficiency Virus, Hepatitis viruses, Creutzfeldt-Jakob disease) and/or of immunological rejection. However, high morbidity associated to donor site, as well as, local pain associated with the invasive harvest procedure extend the hospitalization period.

The alternatives to autologous grafts are allogenic grafts from postmortem human bone tissue and xenografts (non-human animal origin). Their clinical application introduces the possibility of immunological rejection, presents logistics problems and risk of infectious disease transmission to the recipient, which is currently a major concern of physicians, particularly in the case of viral diseases.

The use of synthetic bone grafts, namely, calcium phosphate ceramics, presents itself as the valid reference alternative due to its osteointegration ability. Hydroxyapatite, Ca₁₀(PO₄)₆(OH)₂, and tricalcium phosphate, Ca₃(PO₄)₂, comprise the most commonly used calcium phosphate ceramics in the clinical field owing to their similarity with bone mineral phase, and due to their biocompatibility, bioactivity and osteoconductivity properties.

Several studies attempted to obtain production of synthetic bone grafts with a micro and macroporous structure similar to the micro and macrostructure present in natural mineral bone. These studies focused their objectives in obtaining macrostructure, porosity, pore size, distribution and interconnectivity, which culminates in optimum osteodegeneration. Specifically, microporosity enhances cell adhesion and macroporosity foments bone growth within the bone graft, these factors being decisive for the increase in new bone growth rate locally at the implant site, as described below.

Attaining porosity in bone grafts has comprehended several methodologies, including foam and polymeric sponges-based technology and porogenic agents. In the first case, foams or polymeric sponges are impregnated with a biomaterial suspension and, upon drying, are processed by a thermal process which assures full combustion of the foam or sponge and concomitant formation of open pores. The second technique employs different porogenic substances, such as organic additives and inorganic salts, which upon mixture with the ceramic biomaterial and subsequent appropriate thermal treatment result in porous structures.

However, these methods present recurring disadvantages that are due to non-controlled biomaterial retraction and residue presence after sintering, difficulty in controlling pore dimension, distribution and interconnectivity, and concomitant process reproducibility, presenting consequences at the level of cell colonization of the material.

Additionally, elevated porosity percentages are associated to considerable mechanical resistance reduction compromising the clinical applications of the synthetic bone graft.

On the other hand, in resorbable bone grafts, high porosity and consequent increase in specific surface area resulting in precocious resorption that might compromise bone regeneration due to the absence of physical support, as well as, to the induction of an inflammatory process. Therefore, a compromise between resorption rate and new bone growth rate becomes vital.

In such compromise, and despite the reduction in mechanical resistance associated with the bone graft resorption rate, adequate percentages of micro and macroporosity will overpass those effects via bone cell and blood vessel ingrowth, which are the fundamental features for bone graft osteointegration.

Porosity characterized by pores with diameters equal to 100 μm is the fundamental condition for the capillary vascular growth and for the establishment of osteoprecursor cell-bone graft interactions which are essential for the growth and cell reorganization within the synthetic graft. Micro and macroporosity and pore interconnectivity degree, affect directly the diffusion of gas and nutrients present in physiological fluids, as well as, the metabolic residue removal. As cell growth occurs into the interior of the porous canals the bone graft acts as a structural bridge for bone regeneration.

Document WO0068164 discloses a material with applications as a bone graft, obtained through the reaction between a bioglass and hydroxyapatite (CaO and P₂C₅ in an amount less between 2 and 10% wt % and a source of F-ions), via a sintering process in the presence of a vitreous liquid phase that guaranties bioglass fusion and diffusion into hydroxyapatite structure, which culminates in several ionic substitutions within its matrix. Such phenomenon confers to the bone graft: (a) superior bioactivity, due to the reproduction of bone inorganic phase containing several ionic species that modulate its biological behaviour, and (b) enhanced mechanical properties due to the use of a bioglass CaO—P₂O₅ system that acts as liquid phase during the hydroxyapatite sintering process and by filling the material pores, increases its density, and consequently, its mechanical resistance.

Nevertheless, the bone graft production process described in document WO0068164, does neither result in a final product having a porous structure similar to the one of mineral bone, nor presents a macrostructure (or global geometry) considered ideal for clinical application in bone defects.

Synthetic bone grafts available in the market are usually produced in the form of granules obtained via a dry granulation process, such as described in U.S. Pat. Nos. 5,717,006 and 5,064,436. Briefly, ceramic blocks, previously obtained by pressing and sintering, are submitted to milling and size segregation.

U.S. Pat. No. 5,717,006 discloses a biomaterial for resorption/substitution of bone tissues based on 40 to 75% by weight of β tricalcium phosphate (A) and hydroxyapatite (B), in a ratio A:B of between 20:80 and 70:30, or of calcium titanium phosphate (Ca(Ti)₄(PO4)₆) (C), and 60 to 25% by weight of a liquid phase comprising an aqueous solution of a non-ionic polymer derived from cellulose.

U.S. Pat. No. 5,064,436 discloses a bone prosthetic material consisting of a porous calcium phosphate group based granules having homogeneous sized open cells with an average pore size of 0.01-10 μm, wherein said granules have on average said open cells within a surface area of 10 μm² and the cells are homogeneously distributed and in direct contact with one another.

Despite the structure obtained according to the mentioned methods present porosity, they exhibit irregular and angular geometry susceptible of inducing inflammatory reactions due to differences between individual granule reabsorption rates and eventual tissue damage provoked by edges. Furthermore, the abovementioned geometric irregularity makes the granules unsuitable for controlled drug release, due to the difficulty of a uniform coating with an active pharmaceutical substance.

Documents WO2010021559A1 and Ataíde, L. (2014) Doctorate Thesis disclose the production of a calcium phosphate ceramic spherical pellet through production of the pharmaceutical technology of extrusion and spheronization and a thermal process of sintering in the presence of a vitreous liquid phase. Nevertheless, the bone graft production process described in these documents, does not present a macrostructure considered ideal for clinical application in bone defects. Moreover, these processes do not use aqueous suspensions with biomaterials and porogenic agents and therefore, it is not possible to obtain a calcium-phosphate ceramic granules with micro and macroporosity, with high granulometry range, having higher granules size range with high macroporosity considered ideal for more clinical application in bone defects, enhancing the bone graft osteoconduction and osteointegration of the bone graft. Document WO2019153794A1 discloses the production of hollow spherical particles of bone grafts through technology of extrusion and spheronization with macropores size bet ween 10 μm to 200 μm. According to the mentioned method and despite the resulting structure presenting macroporosity, the presence of a hollow cavity configures a high porosity and increased surface area, that compromises bone regeneration due to the absence of physical support, as well as to the induction of an inflammatory process. Therefore, this process is not able to produce calcium-phosphate ceramic granules with micro and macroporosity, high granulometry range, and macropores size between 50 μm to 600 μm advantageous to improve bone cell and blood vessel ingrowth, which are the fundamental features for bone graft osteointegration.

Due to the abovementioned, the development of implantable biomaterials with porosity that mimics as much as possible the bimodal bone structure (cortical and trabecular) and that presents adequate interconnectivity degree, represents a tremendous challenge.

The present invention relates to a process for producing biomaterials based on hydroxyapatite and bioglass that allow to control the biomaterial retraction and residue presence after sintering, the pore dimension, distribution and interconnectivity in a reproducible manner.

The resulting biomaterials present granulate structure having homogeneous size, whose interconnective porous structure, in the micrometre range, allows for enhanced osteoconductivity and osteointegration with a completely controlled behaviour upon implantation, whilst maintaining good bioactive properties.

### SUMMARY OF THE INVENTION

The present invention relates to a method for producing hydroxyapatite-bioglass materials and products thereof that resemble bone structure and properties, thus being advantageously used in the medical applications, in particular in bone regeneration and reparation. Medical devices, such as bone grafts, prosthetics, implants and derivatives are successfully obtained by the use of these biomaterials.

In a first aspect, the present invention relates to a hydroxyapatite-glass biomaterial according to claim 1.

This biomaterial presents a micro and macroporous structure similar to the one present in natural bone with granulate structure having homogeneous size, whose interconnective porous structure, in the micrometre range, allows for enhanced osteoconductivity and osteointegration with a completely controlled behaviour upon implantation, whilst maintaining good bioactive properties.

This kind of micro and macroporous structure is a fundamental requirement for the occurrence of cell adhesion and bone tissue growth within the material, which constitutes the first essential advantage of this novel biomaterial.

Said biomaterial enhances blood vessels and bone cells migration, allowing bone growth through the interior of the bone substitute, thereby increasing the rate of formation of new bone at the site of implantation.

Additionally, elevated porosity percentages are associated to considerable mechanical resistance reduction compromising the clinical applications of the synthetic bone graft. Cn the other hand, in resorbable bone grafts, high porosity and consequent increase in specific surface area resulting in precocious resorption that might compromise bone regeneration due to the absence of physical support, as well as, to the induction of an inflammatory process.

In a second aspect, the present invention related to medical devices comprising a hydroxyapatite-glass biomaterial according to claim 5.

Medical devices obtained according to the invention resemble the mineral phase of natural bone with excellent mechanical strength and osteoconductivity. Further, they present increased bone fusion, lower morbidity rates of interventions related to bone harvesting and associated limitations, lower risk of infections and rejection by the patient, and good mechanical properties.

In a third aspect, the present invention relates to a process of producing a hydroxyapatite-glass biomaterial according to claim 7.

This process allows to control the biomaterial retraction and residue presence after sintering, the pore dimension, distribution and interconnectivity in a reproducible manner. The reproducibility of the pharmaceutical processes of extrusion and spheronization guaranties the abovementioned characteristics, which in turn translates in a biomaterial whose behaviour is completely controlled and expected upon implantation.

### DESCRIPTION OF THE FIGURES

FIG. 1 represents granules morphology and pore size dimensions of the biomaterial, wherein FIG. 1a is a 65× magnification and FIG. 1b is a 1000× magnification. It is possible to observe pore size, interconnectivity and homogeneous distribution. Macroporous size ranges between 200-600 μm and microporous size ranges between 550 nm and 1.5 μm.

FIG. 2 represents the granulometric distribution of the biomaterial. It is possible to observe that the obtained granules present a size ranging between 150 μm and 6 mm.

### DESCRIPTION OF THE INVENTION

The present invention refers to a hydroxyapatite-bioglass materials, to a process of producing said materials and to medical devices comprising said biomaterials that can be applied in osteoregenerative medicine as a bone graft.

### 1. Hydroxyapatite-Bioglass Materials and Products

### 1.1 Hydroxyapatite-Bioglass Materials

The hydroxyapatite-bioglass material herein disclosed comprise granules based on a P₂O₅—CaO glass system. The bioglass is present in the hydroxyapatite-bioglass mixture in an amount of 1 to 15 wt % of the total weight of the mixture, preferably in an amount of 2 to 10 wt %, more preferably in an amount of 2.5 to 10 wt % of the total weight of the mixture.

In the scope of the present invention, the expression “bioglass” or “biocompatible glass” defines a glass product that does not contain metal ions in an amount nor tolerated or not adequate for use in medical applications, human or veterinary.

Biocompatible glass material comprises the combination of P₂O₅ and CaO in a ratio of 20:80 to 80:20 of molar percentages of each.

Preferably, the biocompatible glass also comprises CaF₂, Na₂O and/or MgO in the following amounts:
CaF₂: 0-20 mol %,
Na₂O: 0-20 mol %,
MgO: 0-20 mol %.

More preferably, the biocompatible glass comprises:
P₂O₅: 60-75 mol %,
CaO: 10-25 mol %,
Na₂O: 0-15 mol %,
CaF₂: 0-15 mol %,
MgO: 0-20 mol %.

The granulometric distribution, analysis, assessment and characterization of the biomaterials of the invention were performed by sieving; the porosity, pore diameter of the macroporous, bulk and apparent density were assessed by means of mercury porosimetry. Macroporous granules surface morphology was assessed by scanning electron microscopy (SEM).

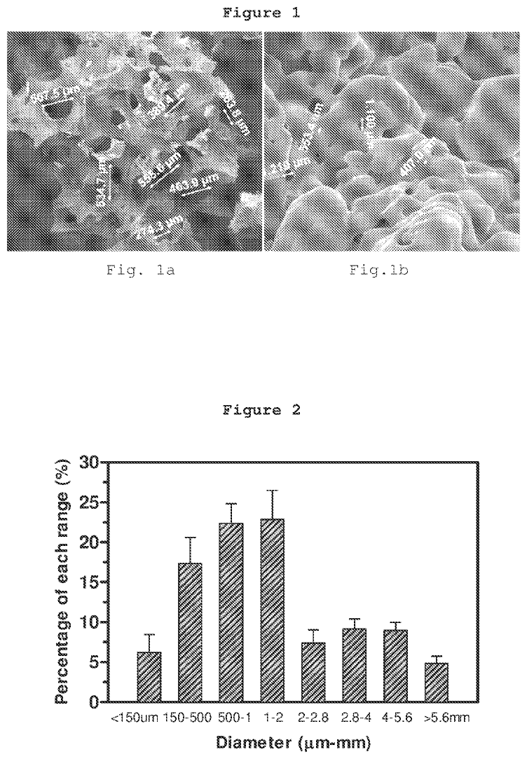

FIG. 1 shows granules morphology and macroporous and microporous interconnective structure of the biomaterial in a particular embodiment of the present invention. According to this embodiment, 25%±2.5% of these granules present a granulometry between 2.0 and 5.6 mm, as shown in FIG. 2.

Some important characteristics of the hydroxyapatite-bioglass materials of the invention are presented in Table 1, where it is possible to observe that is obtained a global porosity of 34-35% with macropore size ranging of 200-600 μm, granule bulk density of 1.413 g/mL, and apparent density of 2.172 g/mL.

TABLE 1
Some Characteristics of hydroxyapatite-bioglass materials

| | |
|---|---|
| Global Porosity (%) | 34.96 |
| Bulk Density (g/mL) | 1.413 |
| Apparent Density (g/mL) | 2.172 |
| Macropore size (μm) | 200-600 |

The material herein disclosed comprise hydroxyapatite-bioglass granules with a global porosity of at least 35 vol %, comprising an intraporosity of at least 20 vol % and an interporosity of at least 20 vol %.

In the scope of the present invention intraporosity refers to the pores existing in the biomaterial.

In the scope of the present invention interporosity refers to pores resulting from the biomaterial packing.

The intraporosity is mainly dependent on the pellet size and on the porogenic agent used, and in the materials of the invention is characterized by the presence of two distinct populations of pores, namely having microporosity with pore size diameter up to 2 μm, with average range size of 550 nm to 1.5 μm, and having macroporosity with pore size diameters superior to 50 μm, with average range size from 100 to 600 μm.

Accordingly, it is possible to obtain a biomaterial with granules present homogeneous size and an interconnective porous structure with the following characteristics:
10 to 20% of the granulometry varies from 150 to 500 μm,
30 to 50% of the granulometry varies from 500 μm to 2 mm, and
40 to 60% of the granulometry varies from 2 to 5.6 mm.

The hydroxyapatite-bioglass biomaterials of the invention present granules with size ranging of 150 μm and 6 mm, wherein the average size varies of 2 and 5.6 mm.

The maximum granulometry is superior to 5.6 mm, in average it varies from 2 mm to 5.6 mm. The granulometry distribution can be characterized as the following:

| | | |
|---|---|---|
| <150 | μm | 6.13% |
| 150-500 | μm | 17.30% |
| 500-1 | mm | 22.31% |
| 1-2 | mm | 22.79% |

| | |
|---|---|
| 2-5.6 | 48.08% |

| | | |
|---|---|---|
| >5.6 | mm | 4.76% |

Hydroxyapatite-bioglass materials can be provided in powder, pellets, granulates or blocks, which can be obtained by any known method in the art suitable to this purpose, in particular having pharmaceutical grade such as conventional processes of extrusion and spheronization.

These materials present several advantages, namely low cost, high reproducibility, high yields and improved characteristics for producing bone grafts.

Concerning the characteristics, the porous structure, cell adhesion promotion and consequent cellular growth, namely, of osteoprecursor cells and blood vessels, induced by the release of ionic species from the biomaterial that culminates in a higher osteointegration and osteoregeneration are the main advantages that can be associated to these materials.

Furthermore, native conformation protein adsorption, present in physiological fluids, at the porous surface of the synthetic bone graft, contributes to an absent immunogenicity and a cellular proliferation increase.

The macroporosity enhances blood vessels and bone cells migration, allowing bone growth through the interior of the bone substitute, thereby increasing the rate of formation of new bone at the site of implantation.

The homogenous size and interconnective porosity of the granules, further allow its application as a controlled pharmaceutical active substance release device, such as growth factors or other growth modulation and bone remodelling agents.

### 1.2. Hydroxyapatite-Bioglass Medical Devices (Bone Grafts)

Hydroxyapatite-bioglass prepared according to the present invention, present improved mechanical properties and can be used in any of dental- and medical-applications for which unmodified hydroxyapatite have been previously used. Examples of fields where hydroxyapatite-bioglass materials are advantageously used are bone implants, or bone fillers where powdered composition is used as a filling material.

The hydroxyapatite-bioglass can also be used in formation of artificial joints in which a coating is applied to at least a part of a metal or alloy joint.

The synthetic bone grafts produced according to the invention are advantageously applicable in osteoregenerative medicine, particularly in the fields of orthopaedic surgery, maxillofacial surgery, dental surgery, implantology and as tissue engineering scaffolds.

### 2. Process for Producing Hydroxyapatite-Bioglass Materials

The process of producing these hydroxyapatite-bioglass materials comprises a first step (a) of preparing an aqueous suspension of hydroxyapatite-bioglass with a porogenic agent, and a second sintering step (b) thus, resulting in a low cost, high yield and reproducible process developed in very controlled conditions.

### 2.1 Hydroxyapatite

A hydroxyapatite compound adequate for use in the present invention can be prepared by precipitation of the product resulting of the reaction between a calcium hydroxide [Ca(OH)₂] suspension in purified water with an aqueous solution of orthophosphoric acid [H₃(PO₄)₂].

10Ca(OH)₂+6H₃(PO)₄→Ca₁₀(PO₄)₆(OH)₂+18H₂O

Preferably, Ca(OH)₂ is present in the water suspension in an amount of 98-100% (wt/v).

Preferably, the H₃(PO₄)₂ is present in the aqueous solution in an amount of 85% (wt/v).

After the preparation of the abovementioned raw material, milling and sieving are performed in order to obtain particles with a granulometry between 10 and 75 μm.

### 2.2 Bioglass

Biocompatible glass adequate for use in the present invention belongs to the P₂O₅—CaO system. It can be prepared by a conventional melting technique with the comb-nation of these two compounds in a ratio of 20:80 to 80:20 of molar percentages of each.

Preferably, the biocompatible glass also comprises CaF₂, Na₂O and/or MgO in the following amounts:
CaF₂: 0-20 mol %,
Na₂O: 0-20 mol %,
MgO: 0-20 mol %.

More preferably, the biocompatible glass comprises:
P₂O₅: 60-75 mol %,
CaO: 10-25 mol %,
Na₂O: 0-15 mol %,
CaF₂: 0-15 mol %,
MgO: 0-20 mol %.

Bioglass preparation can be performed via fusion of a sodium source (e.g., sodium carbonate (Na₂CO₃)), a calcium source (e.g., calcium hydrogenophosphate (CaHPO₄)), a fluor source (e.g., calcium fluoride (CaF₂), magnesium source (e.g., magnesium oxide (MgO)) and a phosphorus source (diphosphorus pentoxide (P₂O₅)) providing the above mentioned amounts of the respective compounds.

After the preparation of the abovementioned raw material, milling and sieving are performed in order to obtain particles with a granulometry having a size ranging from 10 to 50 μm.

### 2.3 Porogenic Agent

Adequate porogenic agent, in the scope of the present invention, is defined as any appropriate substance that that upon sintering, suffers complete calcination not leaving substantially any residue, thus originating a porous structure.

In the scope of the present invention adequate porogenic agents are polyvinyl alcohol (PVA), citric acid (CA), polyvinyl pyrrolidone (PVP), crystalline cellulose, carboxymethylcellulose (CMC). Polyvinyl alcohol (PVA) is the preferred porogenic agent since it produced the best results in forming particles with size and distribution for the intended applications of the invention.

Other adequate porogenic agents include mixtures comprising PVA with at least one of the compounds selected from: cellulose, starch, modified starch, sorbitol, croscarmellose sodium, crospovidone, sodium alginate and lactose, in amounts between 40% and 80 wt % of PVA in the final mixture.

More preferred mixtures comprise PVA and cellulose since PVA contributes for the granule macroporosity and to maintain the solid components of the hydroxyapatite-bioglass mixture in suspension, whilst cellulose contributes for the granule microporosity.

The weight percentage and the type of porogenic agent used is related to the formation of pores and their size thus directly influences not only the porosity of the final biomaterial but of its mechanical strength as well.

A PVA solution adequate for use in the present invention can be prepared by mixing PVA with purified water until full dissolution is achieved, at a temperature of 90° C. to 97° C., to avoid boiling the water. The solution is allowed to cool till room temperature (20° C. to 25° C.).

### 2.4 Hydroxyapatite-Bioglass Materials

Hydroxyapatite, bioglass and porogenic agent as described above are mixed in a formulation comprising up to 10 wt % of bioglass relatively to hydroxyapatite weight, and up to 80 wt % of a porogenic agent relatively to the hydroxyapatite and bioglass powder mixture weight.

Biomaterials according to the invention are prepared by a conventional process, such wet process, employing a mixer, at a rate up to 150 rpm, during an adequate period of time to allow obtaining a homogeneous suspension blend, typically of 15 minutes or more.

The resulting mixture is then dried, preferably in a forced air circulation oven, at a temperature higher to 60° C., preferably between 60-65° C., and for at least 24h. This drying procedure ensures the proper, macroporous structure before the sintering process.

Then, a thermal treatment of the macroporous structure is performed in two phases, where in the first phase the temperature is increased to 400-800° C., preferably to 500-700° C., more preferably to around 600° C., at a rate of approx. 0.1° C./min, more preferably of around 0.5° C./min, during a period of at least 1.5h in order to ensure the complete combustion of the porogenic agent used therein without leaving any substantial residue, whilst originating the porous structure.

The second phase, i.e., the sintering process is performed above 1200° C., preferably at a temperature ranging from 1250° C. to 1350° C., at a heating rate of approx. 4° C./min allowing the bioglass fusion and distribution in the hydroxyapatite matrix in a liquid phase sintering process.

Once the sintering temperature is reached, the sintering treatment in the presence of a vitreous liquid phase occurs during a period of at least 1 h, followed by the posterior natural cooling of the biomaterial to room temperature inside the furnace.

The obtained structure of the hydroxyapatite-bioglass materials thus produced presents several advantages, namely low cost, high reproducibility, high yields and improved characteristics for producing bone grafts.

### EXAMPLES

### Example 1: Hydroxyapatite Preparation

500.00 g hydroxyapatite was prepared by chemical precipitation by using 370.45 g calcium hydroxide (Ca(OH)₂, >98%) and 345.15 g orthophosphoric acid 85 (wt/v) % (H₃PO₄). 9 L purified water was poured in a large appropriated container, calcium hydroxide was added and mixed (Mixer R25) for 15 minutes. Meanwhile, 8 L purified water was poured in an appropriated recipient, orthophosphoric acid was added and the volume was completed with purified water up to 9 L.

The addition of orthophosphoric acid was carried out via peristaltic pump (Minipuls 2) at a constant rate of 150 rpm. The mixture was performed for 4-5 hours, and cleaning of the calcium hydroxide container walls with purified water is required in order to prevent precipitate accumulation.

The pH was adjusted to a value of ≥10.5±0.5 by using a 32% ammonia solution. Thereafter, the container was washed with purified water and the rate of the peristaltic pump was increased to 360 rpm.

The solution was stirred for approx. 1h followed by a resting period for of approx. 16 hours allowing the mixture to ageing.

Hydroxyapatite was then filtered and dried in a forced air circulation oven (Binder), and milled in a planetary mill (Fritsch Pulverizette 6) to achieve a granulometry between 10 and 75 μm.

### Example 2. Bioglass Preparation

0.2 mol of a bioglass with the following nominal composition 65% P₂O₅-15% CaC-10% CaF₂-10% Na₂O (molar %) was prepared, having CaF₂ as fluoride ion source. For that purpose, 2.12 g sodium carbonate (Na₂CO₃), 4.08 g calcium hydrogenophosphate (CaHPO₄), 1.56 g calcium fluoride (CaF₂) and 16.32 g diphosphorus pentoxide (P₂O₅) were weighed and mixed in a platinum crucible. The crucible was placed in a vertical furnace (Termolab) and heated for 1.5 h until a temperature of approx. 1450° C., followed by a dwelling time of 30 minutes. Thereafter, the molten glass was poured into purified water and the glass was allowed to dry.

Then, it was milled in a planetary mill (Fritsch Pulverizette 6) and collected when the granulometry was between 10 and 75 μm.

### Example 3. Porogenic Agent Preparation

20.0 g of PVA (polyvinyl alcohol 8-88, medical grade) was mixed in 900 mL of purified water, for approx. 3h until the PVA is completely dissolved.

A solution comprising PVA and microcrystalline cellulose (Avicel PH101, with a diameter inferior to 50 μm) was prepared by mixing the PVA solution with 10.00 g of microcrystalline cellulose.

### Example 4. Hydroxyapatite-Bioglass Preparation

487.50 g hydroxyapatite, 12.50 g bioglass, 900 mL PVA solution and 10.00 g microcrystalline cellulose prepared as described above were mixed for approx. 22 minutes at 150 rpm in a planetary mixer. The resulting mixture was dried in a forced air circulation oven, at a temperature between 60° C.-65° C., for approx. 24h.

### Example 5. Thermal Treatment and Sintering

The sintering thermal treatment of the macroporous biomaterial was performed at a heating rate of 0.5° C./min, up to 600° C. and kept for a 4 h period, followed by a heating rate of 4° C./min up to 1300° C. being this temperature maintained for approx. 1h. Thereafter, the resulting biomaterial was allowed to cool inside the furnace.

After the sintering, the obtained biomaterial was analysed and evaluated by SEM and mercury porosimetry. FIG. 1 shows its macroporous and microporous interconnective structure, which is in agreement with the porosity assessment.

Further, 25%±2.5% granules of the hydroxyapatite-bioglass macroporous material analysed show granulometry between 2 and 5.6 mm (FIG. 2).

Hydroxyapatite-bioglass macroporous material present a global porosity of 34.96% with macropore-size ranging from 200-600 μm having the macroporous granules a bulk density of 1.413 g/mL, and apparent density of 2.172 g/mL.

1. A hydroxyapatite-bioglass material characterized by comprising hydroxyapatite-bioglass granules based on a P₂O₅—CaO glass system, having:
a bioglass material present in the hydroxyapatite-bioglass mixture in an amount of 1 to 15 wt % of the total weight of the mixture, preferably in an amount of 2 to 10 wt %, more preferably in an amount of 2.5 to 10 wt % of the total weight of the mixture, and the P₂O₅ and CaO ratio in the bioglass material varies from 20:80 to 80:20 of molar percentage of each,
and
the hydroxyapatite-bioglass granules present a global porosity of 34 to 35 vol % being the intraporosity of at least 20 vol % and the interporosity of at least 20 vol %, the macroporous size ranges from 50 μm to 600 μm, preferably from 200-600 μm, and the microporous size ranges from 550 nm to 2 μm, preferably of 550 nm to 1.5 μm, the granules presenting a size ranging of 150 μm to 6 mm, and an overall granulometry from 500 μm to 5.6 mm, preferably of 2 mm to 5.6 mm, wherein:
10 to 20% of the granulometry varies in a range of 150 to 500 μm,
30 to 50% of the granulometry varies in a range of 500 μm to 2 mm, and
40 to 60% of the granulometry varies in a range of 2 to 5.6 mm.

2. A hydroxyapatite-bioglass material according to claim 1 characterized by the bioglass comprising CaF₂, Na₂O and/or MgO in the following amounts:
CaF₂: 0-20 mol %,
Na₂O: 0-20 mol %,
MgO: 0-20 mol %.

3. A hydroxyapatite-bioglass material according to claim 1 characterized by the bioglass comprising CaF₂, Na₂O and/or MgO in the following amounts:
Na₂O: 0-15 mol %,
CaF₂: 0-15 mol %,
MgO: 0-20 mol %, and
P₂O₅: 60-75 mol %,
CaO: 10-25 mol %.

4. A hydroxyapatite-bioglass material according to claim 1 characterized by being in the form of a powder, pellets, granulates or blocks.

5. A medical device characterized by comprising a hydroxyapatite-bioglass material as described in claim 1.

6. A medical device according to claim 5 characterized by being a bone implant or a bone filler.

7. A process for producing a hydroxyapatite-bioglass material as defined in claim 1, characterised by comprising the following steps:
a) Providing a mixture comprising hydroxyapatite, bioglass and a porogenic agent, being the porogenic agent a mixture of polyvinyl alcohol (PVA) with one of the selected agents: citric acid (CA), polyvinyl pyrrolidone (PVP), microcrystalline cellulose, carboxymethylcellulose (CMC), starch, modified starch, sorbitol, croscarmellose sodium, crospovidone, sodium alginate and lactose, and wherein the PVA is present in an amount of 40 to 90 wt % of PVA in the final mixture, preferably of 60 to 80 wt % of PVA in the final mixture, and
b) Performing a thermal treatment by sintering to the mixture of (a).

8. A process according to claim 7 characterized by the porogenic agent comprising PVA and at least one of the compounds selected from microcrystalline cellulose, starch, modified starch, sorbitol, croscarmellose sodium, crospovidone, sodium alginate and lactose, wherein the PVA is present in an amount of 40 to 90 wt % of PVA in the final mixture, preferably of 60 to 80 wt % of PVA in the final mixture.

9. A process according to claim 8 characterized by the porogenic agent being a mixture of PVA and microcrystalline cellulose, wherein the ratio of each varies from 40:60 to 20:80, preferably from 25:75 to 50:50.

10. A hydroxyapatite-bioglass material as described in claim 1 characterized by being applicable in the medical area.

11. A hydroxyapatite-bioglass material as described in claim 1 characterized by being applicable in the osteomedical area.

12. A hydroxyapatite-bioglass material as described in claim 1 characterized by being applicable in bone regeneration and bone reparation techniques.
